(19) United States
(12) Patent Application Publication
(10) Pub. No.: US 2004/0068125 A1
(43) Pub. Date: Apr. 8, 2004

(54) Foundation building system with antiseismic plates

(76) Inventor: Angel Castillo Senal, Zaragoza (ES)

Correspondence Address: SCHIFF HARDIN, LLP, PATENT DEPARTMENT, 6600 SEARS TOWER, CHICAGO, IL, 60606-6473, US

(21) Appl. No.: 10/451,312

(22) PCT Filed: Dec. 17, 2001
(86) PCT No.: PCT/ES01/00487

(30) Foreign Application Priority Data
Dec. 19, 2000 (ES) P200003040

(51) Int. Cl. C07D 307/78; C07D 307/87; C07D 307/93

(52) U.S. Cl. 549/467

(57) ABSTRACT

The invention aims at isolating the foundations and the remaining part of the building from the earthquake. The antiseismic plates (2) are hermetic containers divided into two parts. Vacuum has been produced in part “B” so that most of the longitudinal or primary seismic waves propagating through the solids and fluids are cancelled. A pressurized liquid has been introduced into part “A” to cancel the transversal or secondary waves that propagate through the solids. “F1” is the lateral force of the surface seismic wave acting upon the plate and producing a pressure on the liquid pressing against the base (1) with nil results. “F4” is the decompensation of the faces pressing against the ground. The invention can be used in all types of foundations and large surfaces to preserve infrastructures, etc. It can alternatively be used for conical bases, buried walls, beam supports, etc.

The sector of the art to which the present invention is related, is that of earthquake-resisting structures and the part of Geophysics which deals with Seismology

### BACKGROUND OF THE INVENTION

The state of the art considers earthquakes to be practically unpredictable. The main causes which produce an earthquake are plate tectonics, and there immediate effects are elastic forces, which originating at the focus or hypocentre, traverse the Earth (longitudinal or primary waves and transverse or secondary waves).

When these forces reach the surface, at the earthair or earth-water interface, they produce surface forces of different intensity or wavelength, Love waves, Rayleigh waves, long or L waves).

The surface waves are the most destructive, acting on structures in both the horizontal and vertical directions endeavouring to displace them. This is propagated through the foundations to the rest of the structure, producing tremors and twisting.

Investigations carried out up to now, deal with the causes of earthquakes, and all that can contribute to the prediction thereof in order to avoid disasters (in 1975, the Chinese were able to predict an earthquake in the Haicheng region). Moreover, attempts are being made to dampen their destructive effects, designing buildings in a pyramidal, symmetrical form, with the lifts in the centre, and reinforcing the structures, even with concrete enriched with carbon fibres.

Another field of research is attempting to achieve ductile materials and in turn resistant, which will absorb or dampen the seismic waves.

The ideal situation would be to dampen or destroy the seismic wave before it has an effect on the foundations. Along these lines, investigations carried out with electro-rheological materials allow to foresee that in the near future foundations will be able to be made taking these properties into account.

These materials have the quality that whilst a current of electricity is passed through the mass they remain in a solid state—rigid, and when it stops, it changes in a matter of milliseconds to a gelatinous state, which absorbs the seismic waves better.

In Spain, the valid Regulation is: Seismic Resistant Construction Standard, NCSE 94

### DESCRIPTION OF THE INVENTION

In order to give a greater understanding of the invention, the most relevant concepts that have been taken into consideration are stated briefly:

1.—All seismic wave forces are inter-reactive by contact, needing an ideal medium for their propagation.

2.—The longitudinal or primary seismic and similar waves act like sound waves, and are transferred through solids and liquids.

3.—The transversal or secondary and similar waves are propagated only through solids.

4.—Incompressibility of liquids.

5.—A contact interactive force cannot be propagated through a vacuum.

6.—A force line cannot be transmitted through a liquid.

7.—Pascal's Theorem has been taken into consideration in its full context.

8.—The proportions have been deliberately exaggerated in the drawings for a better explanation.

9.—Only direction, sense and point of application have been considered in the forces.

The aim of the invention is to isolate the foundations from the elastic forces of earthquakes, by means of anti-seismic plates, thus avoiding that the static balance of the constructed system is destroyed (buildings, bridges, etc.) and so that the seismic waves are not propagated to the rest of the construction.

The anti-seismic plates, FIG. 1—are receptacles preferably having a square shape and of variable thickness, which are divided into two parts—FIG. 2—.

In part (A), a liquid or semi-liquid has been introduced under a small amount of pressure. This is for the purpose of converting the seismic wave under pressure and destroying its line of force.

In part (B), the vacuum has been produced to prevent the propagation of the longitudinal waves, given that these are propagated through solids and fluids, but not through a vacuum.

The anti-seismic plates have to support large pressures. In part (A) the incompressibility of liquids has been taken into consideration, and in part (B) (where the vacuum is produced) some separators (2) have been fitted between the faces (1 and 3), preferably having a spherical shape to obtain the greatest resistance possible with the minimum contact.

In FIG. 2, it is highlighted that the face (1) and face (3) have a small amount of flexibility. This is so that the mechanical force of the seismic wave will apply pressure on the liquid in the receptacle (A).

Face (4) of FIG. 2 will always be in contact with the construction system, as can be seen in FIG. 3.

Following the order of frequency of the elastic force of the earthquake, it first acts on face (1) FIG. 2-, this puts force on the separators (2), and these on face (3), this on the liquid of receptacle (A), where the force is converted into pressure on face (4), an this face on the construction system. In FIG. 3 we have the correct assembly of the anti-seismic plates (2), in the base of the plate (1) and its sides or perimeter. The perimeter plates (if it is not a single plate that surrounds the base plate), will be joined from one to the other by the part of receptacle (A) so that the liquid reacts for the whole of the perimeter at the same moment. Force (F1) represents the static force of the building, force (F2) is the reaction of the ground, this force can be substituted by any other force that is equal or less (earthquake) without it breaking the static balance.

Force (F3)—FIG. 3—is considered as the superficial seismic force, this force applies pressure on the liquid (A), this in turn exercises a pressure that surrounds the plate and tries to compress it. These forces are equal and opposing hence, it becomes cancelled out.

Force (F4) would be the imbalance of the faces. Force (F3) would be the action and force ‘F4’ the reaction.

### ADVANTAGES

The advantages to be highlighted are:

The damping or cancelling of seismic waves is achieved, avoiding tremors and twisting in the buildings, which allows different projects to be carried out.

An attempt is made to avoid the psychological effect of the earthquake on people who are in the buildings, which is where it is felt.

Its application can be adapted to any construction, which allows it to cover large areas so as to protect infrastructures, water, gas pipes, etc.

It can be applied to dampen vibratory movements of machines, sound waves, etc., likewise for the support of beams or decking for bridges, etc.

### DESCRIPTION OF THE DRAWINGS

FIG. 1.—Elevation

FIG. 2.—Cross-section of the anti-seismic plate:

(A) space occupied by the liquid; (B) space where the vacuum has been made;

(1) external face in contact with the ground; (2) separators of faces (1) and (3);

(3) separation between the faces (A) and (B); (4) external face in contact with the construction system.

FIG. 3.—(1) base plate and pillar; (2) plates; (F1) static force; (F2) reaction force of the ground; (F3) superficial force of the earthquake; (F4) force from imbalance of the faces.

FIG. 4.—Cut elevation in order to be able to appreciate the plates.

FIG. 5.—Section of FIG. 4:

(1) pillar; (2) plates; (3) base plate; (4) box that surrounds the base plate; (5) perimeter wall that surrounds the building; (6) insulating concrete; (7) insulating float concrete for the support of the plates: (8) reinforced base plate for building bracing; (9) air or fill cavity; (10) bedding or steel reinforced plate with expansion joints for the walls and pillars; (11) ground.

FIG. 6.—(1) conical base plate and pillar; (2) plates; (F1) lateral force of the earthquake; (F2) force from imbalance; (R1) and (R2) components of (R3); (R3) result in a downward direction.

FIG. 7.—underground wall:

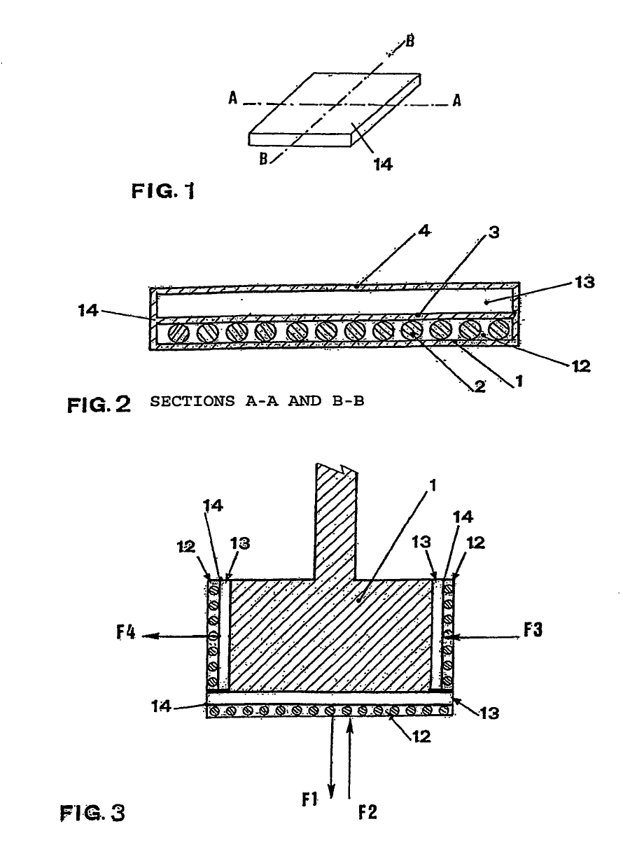

(1) concrete fill; (2) plates; (3) ground; (F1) earthquake force.

By way of description but without limitation, three forms of the preferred construction are detailed, as the characteristics are different.

In the construction of buildings and similar constructions, a double anti-seismic barrier is highlighted that is made up of the perimeter wall and the base of the building, with the anti-seismic plates being as big as possible, communicated from one to the other (in a closed circuit) for the purpose of the liquid reacting at the same moment. It will be made up of a reinforced slab to brace the entire building and to protect the base plate if considerable cracks are produced in the ground.

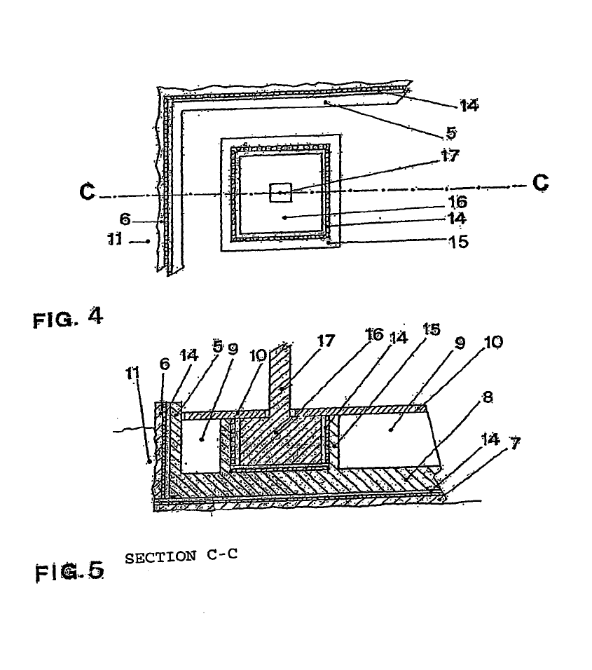

A preferred form of the construction is shown in FIGS. 4 and 5:

After emptying the land, the perimeter wall (6) is straightened with insulating concrete and also the base of the foundations (7), the anti-seismic plates (2) are installed and then the iron corresponding to the reinforced slab (8) the walls (5) and that which is surrounding the base plate or box (4). The slab (8) is concreted, subsequently the plank moulding for or the walls (5) is made and the box that will contain the base plate (4). Once concreted and the panel forms removed, the anti-seismic plates (2) are installed in the interior of the box, thus protecting the base plate.

One single plate will be installed on the base of the base plate, and for the perimeter (in this case four sides, FIG. 4) will also be a single plate (2) FIG. 4, in the case of there being four plates, the part corresponding to the liquid (A) FIG. 2 would have to be communicated in a closed circuit.

The iron grid of the base plate (3) is installed, and the iron of the pillar (1) and then concreted. The spaces (9) can be an air cavity or be filled with gravel or something similar in order to achieve static strength. The bed or sole (10) will have an expansion joint with the walls and with the pillars. The ground is represented as N^{o} (11) in FIGS. 4 and 5.

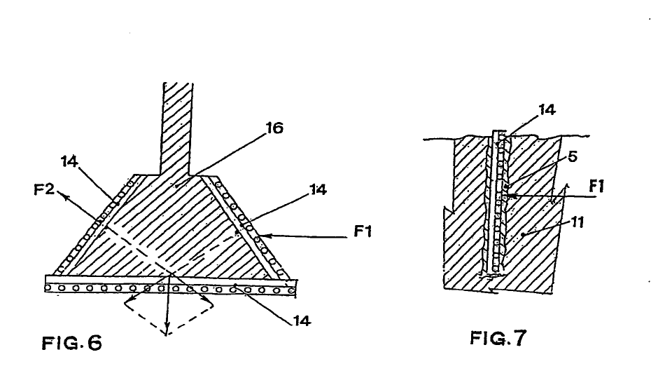

In FIG. 6, a base plate is shown which is worth mentioning because of its conical shape. This is specially suitable in projects for building with low static weight or isolated base plates (bridges, elevated roads, etc.). It can have other shapes such as a pyramid or cylinder, etc.

The aim of these types of base plates is to present the lowest surface possible to the superficial seismic waves and to form angles of refraction, in this way making it easier for the force to be propagated to where there is least resistance offered, specifically surrounding the base plate (1) in addition to obtaining a resultant force that pushes downwards or anchoring, which is added to the static force.

In a diagrammatic manner, the following explanation is stated only by way of taking in the idea of its qualities.

Force (F1)—FIG. 6, is the force of the superficial seismic waves that act on the base plate (1), part of this force surrounds the base plate and is propagated upwards on meeting the least resistance, and the other part of the force acts on the seismic plate (2), producing a pressure in the liquid, which on applying pressure in a perpendicular way to the walls which contain it gives us the results (R1) and (R2) (it is understood that two opposing points of the perimeter have been considered where the resultants are formed).

From the resultants (R1) and (R2), we obtain another resultant called (R3) which produces a vertical force downwards.

The excavation for the base plates is normally in a cubic form; hence, the rest of the space is filled with harsh concrete to obtain a static force on the plate that counteracts the force (F2), which if broken down produces two forces, one horizontal and the other vertical in an upward direction.

The purpose of the underground walls—FIG. 7—is to protect the constructions already made from the superficial seismic waves, these being the most destructive.

The seismic force (F1),—FIG. 7—is propagated through the ground (3) and the harsh concrete fill (1), acting on the plates (2) and these in turn on the ground in a uniform manner.

The efficiency of these walls is relative, as although they cancel out the Love waves, they can allow the rest of the seismic waves, on having different wave lengths, to transfer part of the force to underneath the wall. This method of construction would require studies of distance, depth, etc.

It is thought that this idea of underground walls would be more effective or practical using electro-rheological materials if the costs will allow. The carrying out of this would be very

1. FOUNDATION CONSTRUCTION SYSTEM WITH ANTI-EARTHQUAKE PLATES, characterised in that the foundations are isolated from the seismic forces or any other similar force, before these can act on the foundations, avoiding their propagation to the rest of the structure, be they buildings, bridges, etc., by means of anti-seismic plates.

2. FOUNDATION CONSTRUCTION SYSTEM WITH ANTI-EARTHQUAKE PLATES, characterised by the adequate emplacement of hermetic receptacles divided into two parts—FIG. 2. In one of the parts a liquid under pressure has been introduced for the purpose of converting the elastic force of the earthquake into pressure and so destroy its line of force (A). In the other part a vacuum has been created in order to dampen the longitudinal or similar waves which are propagated through solids and fluids (B).

3. FOUNDATION CONSTRUCTION SYSTEM WITH ANTI-EARTHQUAKE PLATES, characterised in that the plates can be independent or intercommunicated with each other, have different shapes and measurements, be emplaced individually or stacked employing the most appropriate materials for their fabrication. The arrangement of the hermetic compartments in the plates can also be changed, in which for example, a compartment of type “B” (vacuum), could be left sandwiched between two compartments of type “A” (liquid). In addition, the possibility is also envisioned of fabricating plates with a single compartment (that of liquid), for example, for structure which extend over large surfaces—like roads, with the aim of reducing costs.

4. FOUNDATION CONSTRUCTION SYSTEM WITH ANTI-EARTHQUAKE PLATES, characterised in that, in the isolated footings, it is considered that their shape contributes to their effectiveness when they are cylindrical, pyramidal, conical, etc.,—FIG. 6—since they offer less lateral surface area to the seismic wave, create angles of refraction and obtain a vertical resultant force acting downwards.

5. FOUNDATION CONSTRUCTION SYSTEM WITH ANTI-EARTHQUAKE PLATES, characterised by the construction of buried walls to protect the structures already implemented from surface waves—FIG. 7.
